(19) United States
(12) Patent Application Publication
(10) Pub. No.: US 2005/0096661 A1
(43) Pub. Date: May 5, 2005

(54) Insulated battery pack and method of manufacturing same

(75) Inventors: Wade P. Farrow, Fort Worth, TX (US); Sreenivas Chowdiah, Plano, TX (US)

Correspondence Address: HAYNES AND BOONE, LLP, 901 MAIN STREET, SUITE 3100, DALLAS, TX, 75202, US

(73) Assignee: Medtronic, Inc., Minneapolis, MN (US)

(21) Appl. No.: 10/698,636

(22) Filed: Oct. 31, 2003

(51) Int. Cl. A61B 17/00; A61B 17/14; A61B 17/16

(52) U.S. Cl. 606/79; 606/167

(57) ABSTRACT

A battery pack in which a housing is formed by a thermal insulative material, and one or more batteries are disposed in the housing so that the life span of the batteries are not significantly compromised when the battery pack is exposed to relatively high temperatures.

### BACKGROUND

This invention relates to surgical instruments, and more specifically, to a power supply such as a battery pack for use in surgery-related environments.

Many medical tools require a portable power source to supply power to the tool during the medical procedure. The power source is often in the form of a battery pack that is directly attached to the tool and consists of one or more rechargeable batteries enclosed in a housing.

The battery pack, like the tool, must be sterilized before each medical procedure, and this is often done by autoclaving in which the battery pack is placed in a pressurized, steam-heated vessel. However, since the capacity of each battery decreases predictably and repeatedly with each sterilization cycle, the life span of the battery can be shortened-as much as 80 per cent. As a result, premature battery failure can occur during surgery.

One technique that has evolved in an attempt to eliminate this problem involves sterilizing only the housing of the battery pack, and then inserting the batteries in the housing without breaching the sterility of the housing. However, techniques of this type incur a relatively high risk of contamination, take up additional time during operating room setup prior to surgery, and require one sterile and one non-sterile person to perform the technique.

Therefore, what is needed is a battery pack that can be exposed to relatively high temperature environments, such as those encountered during sterilization, without significantly reducing the power output and the life span of the batteries, and without incurring the above problems.

### SUMMARY

A surgical system, a power supply such as a battery pack, and a method of manufacturing the same are disclosed. In one embodiment, the surgical system comprises a tool for cutting bone, tissue, or otherwise used in-a surgical procedure,-an electric motor for driving the tool, and a selectively attachable battery pack. The battery back comprises an outer housing, an inner housing disposed in the outer housing, at least a-portion of the inner housing being formed by a thermal insulative material, and at least one battery disposed in the inner housing.

In another embodiment, a battery pack for use with an electric-powered surgical instrument is disclosed. The battery pack comprises a housing, at least a portion of which is formed by a thermal insulative material, and at least one battery disposed in the housing for providing electric power to the surgical instrument.

In yet another embodiment, a battery pack for use with a surgical instrument having an electric motor is disclosed. The battery pack comprises a housing selectively connectable to the surgical instrument, at least one battery disposed in the housing, and a thermal insulative material extending around the battery.

In yet another embodiment, a battery pack for use in a surgical instrument is disclosed. The battery pack comprises a housing, at least one battery disposed in the housing and in electrical communication with the surgical instrument, and a plate or panel disposed between the battery and the housing, at least a portion of the plate or panel being formed by a thermal insulative material.

In yet another embodiment, a battery pack for selective attachment to a powered surgical instrument is disclosed. The battery pack comprises a housing comprising two spaced walls forming a vacuum space therebetween, and at least one battery disposed-in the housing, the vacuum space-thermally insulating the battery.

In yet another embodiment, battery pack for use with a medical instrument, the battery pack comprising a sealed enclosure placed under a vacuum, and at least one battery disposed in the housing, the vacuum thermally insulating-the battery.

In another embodiment, a method of manufacturing a battery pack for use with a surgical instrument is, disclosed. The method comprises forming at least a portion of a housing of a thermal insulative material, and disposing at least one battery in the housing.

In some embodiments, the thermal insulative material is such that the life span of the battery is not significantly compromised when exposed to a temperature above its rated temperature, such as occurs during an autoclave procedure.

In some of the embodiments, the thermal insulative material from the group consisting of: a silica aerogel; silicone chemical vapor deposition onto the surface of ceramic fabric; fibers formed by a carbon, or silicon carbide, and oxide and impregnated with ceramic material; a polymide foam; a nanoporous silica coating on a polymer film; a hydrous calcium; fused silica; and a composite of vermiculite, fumed silica, hardening agent, and drawn fiber.

One advantage of one or more of the present embodiments is that a battery pack can be exposed to relatively high temperature environments, such as those encountered during sterilization or autoclave, without significantly reducing the power output and the life span of the batteries, and without incurring the above problems.

Additional advantages will be apparent upon review of the attached drawings and the following detailed description. It is understood, however, that several embodiments are disclosed and not all embodiment will benefit from the same advantages.

### BRIEF DESCRIPTION OF THE DRAWINGS

FIG. 1 is a side view of a surgical instrument with an attached power supply, according to an embodiment of the present invention.

FIG. 2a is an isometric view of a battery pack housing utilized in a battery pack according to an embodiment of the invention.

FIG. 2b is an isometric view of an insulation housing utilized in the battery pack of the above embodiment.

FIG. 3 is an isometric view of the assembled battery back with a portion cut away.

### DETAILED DESCRIPTION

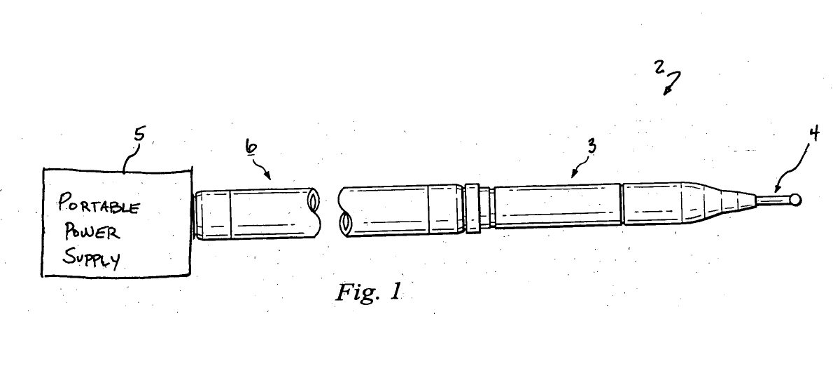

Referring to FIG. 1 of the drawings, the reference numeral 2 designates, in general, a surgical system according to at least one embodiment of the present invention. The surgical system has utility for various applications in which it is desired, including but not limited to:
1. Arthroscopy—Orthopaedic
2. Endoscopic—Gastroenterology, Urology, Soft Tissue
3. Neurosurgery—Cranial, Spine, and Otology
4. Small Bone—Orthopaedic, Oral-Maxiofacial, Ortho-Spine, and Otology
5. Cardio Thoracic—Small Bone Sub-Segment
6. Large Bone—Total Joint and Trauma
7. Dental and other applications

The surgical system 2 includes a motor 3 for driving a tool 4, a power supply 5 for providing energy to the motor, and a electro/mechanical connection 6 between power supply and the motor. In one embodiment, the power supply 5 is a battery pack that selectively attaches directly to the motor 3. In other embodiments, power supply 5 is permanently connected to the motor 3. Furthermore, in some embodiments, the connection 6 represents an elongated electrical cord.

Referring to FIG. 2a, the reference numeral 10 refers, in general, to a housing forming a portion of a battery pack according to an embodiment of the invention. The housing 10 has an enlarged base portion 10a and a stem portion 10b extending from the base portion. The housing 10 can be fabricated of any conventional material including a metal or a plastic such as polyetherimide, polyetherether-ketone, polysulfone, polycarbonate, polyethersulfone/polyarylsulfone, polyphenylene sulfide, acrylonitrile-butadiene-styrene, or liquid crystal polymer. The housing 10 is preferably formed by two half portions that are attached in any known manner so that they can be manually split apart to permit access to the interior of the housing, for reasons to be described.

A clipping mechanism 12 is provided on the stem portion 10b to enable the housing 10 to be connected to a hand held tool (not shown). Since the clipping mechanism 12 is conventional, it will not be shown or described in any further detail.

Referring to FIG. 2b, an insulation housing 14 is provided that is shaped similarly to the housing 10 but is slightly smaller so as to extend in the interior of the housing 10 with a relatively small clearance. The insulation housing 14 forms a complete enclosure and can be formed by two half portions that are attached in any-know manner so that they can be split apart to permit access to a chamber defined in the interior of the housing.

The insulation housing 14 is fabricated, at least in part, from a relatively high thermal insulative material so as to create a thermal barrier, for reasons to be described. To this end, the housing 14 can be fabricated from one or more of the following materials:
1. A silica aerogel of silica, organic aerogels, and/or carbon-particle silica aerogels, with the optional addition of a relatively small percentage of carbon black (such as approximately 9%) and/or the application of a relatively slight vacuum (such as approximately 50 Torr) to lengthen the free mean path of gas-relative to pore diameter.
2. A silicone chemical vapor deposition onto the surface of ceramic fabric, such as silicon carbide, aluminum oxide, or zirconium oxide. Other materials that can be deposited on the surface of the ceramic fabric include advanced flexible reusable surface insulation, tailorable advanced blanket insulation, fibrous refractory composite insulation, and an advanced enhanced thermal barrier.
3. Fibers formed by a carbon, or silicon carbide, and oxide (such as fibers marketed under the name “Nextel⇄ by the 3M company of Minneapolis, Minn.) and impregnated with ceramic material using pre-ceramic polymer impregnation and pyrolysis, or by an enhanced vapor infiltration process. The matrix used can be refractory carbides, nitrides, borides such as SIC, HfC, TaC, BN, Si3N4, or HfB2 and alloys of those materials.
4. A polymide foam, designated as “TEEK” by NASA's Langley Research Center and marketed under the name “Solrex” by the Sordal company of Holland, Mich. This foam can be combined with hollow microspheres and/or paper manufactured by the above Sordal company, under the name “Sordal”.
5. A relatively thin nanoporous silica coating on a polymer film substrate deposited with aluminum by CVD, or by a conventional sputtering technique
6. A hydrous calcium silicate marketed under the name “Thermo-10-gold” by the Johns Manville Company of Denver, Colo.
7. A syntactic foam-based insulation composed of an adhesive layer, an insulation layer, and an outer membrane barrier/protective coating., such as the type marketed by the Composite Technology Development company of Lafayette, Colo.
8. Fused silica that consists of spherical molecules in point contact so that the interstices, or micropores, between the silica particles trap air and prevent heat transmission by convection.
9. A composite of vermiculite, fumed silica, hardening agent, and small amounts of drawn fiber.

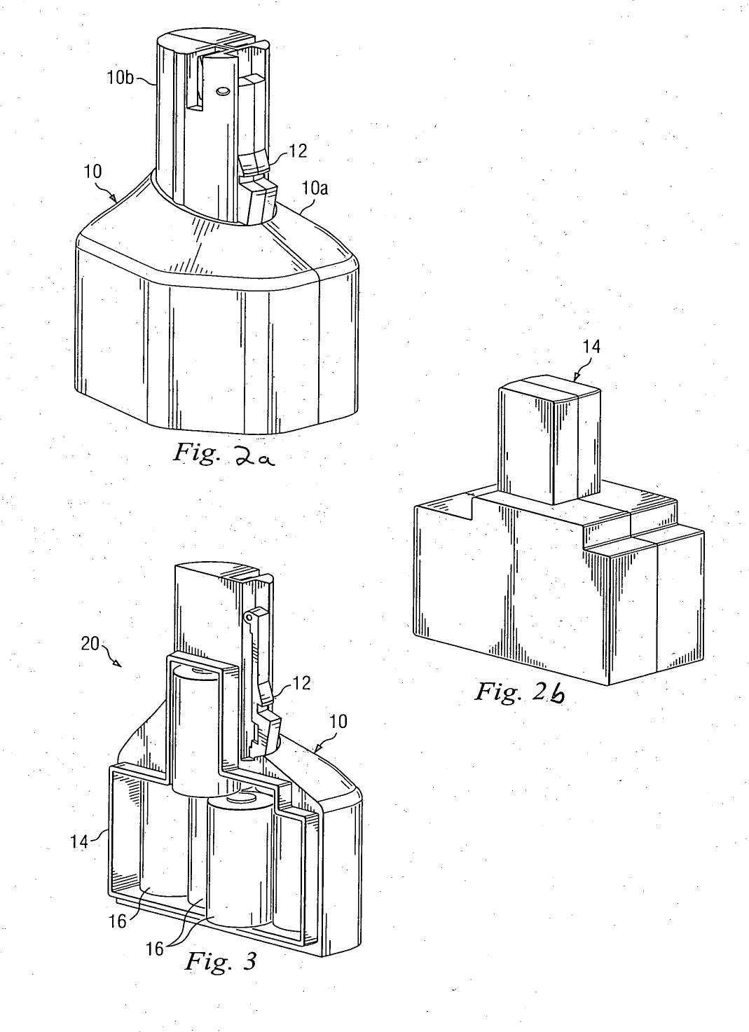

FIG. 3 depicts the insulation housing 14 extending in the interior chamber of the housing 10 along with six batteries 16 disposed in the insulation housing-to form a battery back, referred to, in general, by the reference numeral 20.

Each battery 16 is conventional and, as such, has a positive and negative terminal. Although not shown in the drawings in the interest of clarity, it is understood that electrical circuitry is provided in the interior of the insulation housing 14 that is connected to the terminals of each battery 16. The circuitry includes a circuit board located in the housing 10 and two (positive and negative) output terminals located in the stem portion 10b of the housing 10, for contacting appropriate terminals on the tool to be driven by the battery pack. Since this type of electrical circuitry is conventional, it will not be described in further detail.

Each battery 16 can be in the form of a chargeable battery utilizing conventional chemical elements, such as NiCad, Li Ion, HCL, micro fuel cell, lead acid, or the like, to permit electrical power to be stored. In this context, it is understood that the batteries can be recharged individually, or as a group, by conventional charging apparatus.

To assemble the battery pack 20, the housing 14 is opened in the manner discussed above, the batteries 16 are placed in the housing as shown in FIG. 3, and the above-mentioned electrical circuitry is connected to the batteries. The insulation housing 14 is closed, the housing 10 is opened in the manner described above and the insulation housing 14 is placed therein, as shown in FIG. 3. As an option, the insulation housing 14 can be sealed to the inner surfaces of the corresponding walls of the housing 10 by any appropriate material such as glass fiber, epoxy, or aluminum foil tape in accordance any standard manufacturing techniques.

The housing 10 is then closed and the battery pack 20 is connected to a tool (not shown) by the clip mechanism 12. The batteries 16 output a voltage based on the cumulative voltage of the batteries, to drive the tool, in a conventional manner.

The insulation housing 14 insulates the battery pack 20 from relatively high temperatures: which would normally significantly reduce the normal life span of the batteries. For example, if the tool is used for medical procedures, the battery pack (and the tool) would have to be sterilized between procedures, thereby exposing the batteries 16 to repeated, relatively high temperature sterilization cycles, which can be as much as 70 degrees C. above the upper temperature rating of one or more of the types of batteries mentioned above. However, the insulation housing 14 has sufficiently high thermal insulative properties so as to create a thermal barrier which is sufficient to insure that the life span of the battery pack 20 is not significantly compromised as a result of this exposure.

According to an alternate embodiment, the insulation housing 14 is eliminated and one or more of the insulative materials listed above is wrapped around each battery 16, or group of batteries, when practicable, and secured to the batteries as needed while leaving the above-mentioned terminals of the battery exposed.

According to another embodiment, one or more of the insulative materials listed above is sprayed on the batteries 16a-16e when practicable, while leaving the above-mentioned terminals of the battery exposed. This embodiment could be used with or without the insulation housing 14.

According to another embodiment, the insulation housing 14 is eliminated and -all or part of the housing 10 is fabricated with one or more of the insulative materials listed above.

According to another embodiment, the insulation housing 14 is eliminated and one or more plates or slabs are fabricated from one or more of the insulative materials set listed above, and placed in the housing 10 between the batteries and the inner walls of the housing.

According to another embodiment, the housing 10 is provided with two spaced walls to form a vacuum space between the walls. This embodiment could be used with or without the insulation housing 14.

According to another embodiment, the housing 10 is in the form of-a sealed enclosure that is placed under a vacuum so as to limit the thermal conductivity from outside the housing to the batteries 16. This embodiment could be used with or without the insulation housing 14.

In each of the above embodiments the thermal barrier created by the insulative material maintains the temperature of the batteries within the manufacturer's recommend range, even when the batteries are subjected to high temperature environments such as repeated sterilization cycles. Thus, the normal life span of the batteries is not compromised.

It is understood that modifications and variations may be made in the forgoing without departing from the scope of the invention. For example, the battery packs of the above embodiments are not limited to use with medical tools but can be used in any environments that subjects them to relatively high temperatures. Also, the number and type of batteries used can vary. Further, the particular shape and design of the insulation housing 14 can be varied. Also, all, or a portion of the inner housing may be fabricated from an insulation material. Moreover, the housing 10 may be adapted to receive the housing 14, and the housing 14 may be adapted to receive the batteries 16 in a manner other than those discussed above. For example, a wall of the housing 10 or 14 can be provided with a hinged or removal panel to permit access to its interior by the housing 14 and the batteries 16, respectively.

Those skilled in the art will readily appreciate that many other modifications are possible in the exemplary embodiments discussed above without materially departing from the novel teachings and advantages of this invention. Accordingly, all such modifications are intended to be included within the scope of this invention as defined in the following claims. In the claims, means-plus-function clauses are intended to cover the structures described herein as performing the recited function and not only structural equivalents, but also equivalent structures.

1. A surgical system comprising a tool for cutting bone or other tissue, an electric motor for driving the tool, and a selectively attachable battery pack, the battery back comprising an outer housing, an inner housing disposed in the outer housing, at least a portion of the inner housing being formed by a thermal insulative material, and at least one battery disposed in the inner housing.

2. The surgical system of claim 1 wherein the thermal insulative material is such that the life span of the battery is not significantly compromised when exposed to a temperature above its rated temperature.

3. The surgical system of claim 1 wherein the thermal insulative material is such that the life span of the battery is not significantly compromised when exposed to a temperature that is as much as 70 degrees C. above its rated temperature.

4. A battery pack for use with an electric-powered surgical instrument, the battery pack comprising a housing, at least a portion of which is formed by a thermal insulative material, and at least one battery disposed in the housing for providing electric power to the surgical instrument.

5. The battery pack of claim 4 wherein the thermal insulative material is such that the life span of the battery is not significantly compromised when exposed to a temperature above its rated temperature.

6. The battery pack of claim 4 wherein the thermal insulative material is such that the life span of the battery is not significantly compromised when exposed to a temperature that is as much as 70 degrees C. above its rated temperature.

7. A battery pack for use with a surgical instrument having an electric motor, the battery pack comprising a housing selectively connectable to the surgical instrument, at least one battery disposed in the housing, and a thermal insulative material extending around the battery.

8. The battery pack of claim 7 wherein the thermal insulative material is wrapped around the battery.

9. The battery pack of claim 7 wherein the thermal insulative material is sprayed on the battery

10. The battery pack of claim 7 wherein the thermal insulative material is such that the life span of the battery is not significantly compromised when exposed to a temperature above its rated temperature.

11. The battery pack of claim 7 wherein the thermal insulative material is such that the life span of the battery is not significantly compromised when exposed to a temperature that is as much as 70 degrees C. above its rated temperature.

12. A battery pack for use in a surgical instrument, the battery pack comprising a housing, at least one battery disposed in the housing and in electrical communication with the surgical instrument, and a plate or panel disposed between the battery and the housing, at least a portion of the plate or panel being formed by a thermal insulative material.

13. The battery pack of claim 12 wherein the thermal insulative material is such that the life-span of the battery is not significantly compromised when exposed to a temperatures above its rated temperature.

14. The battery pack of claim 12 wherein the thermal insulative material is such that the life span of the battery is not significantly compromised when exposed to a temperature that is as much as 70 degrees C. above its rated temperature.

15. The battery pack of claim 12 wherein thermal insulative material is selected from the group consisting of:
a. a silica aerogel,
b. silicone chemical-vapor-deposition onto the surface of ceramic fabric,
c. fibers formed by a carbon, or silicon carbide, and oxide and impregnated with ceramic material,
d. a polymide foam,
e. a nanoporous silica coating on a polymer film,
f. a hydrous calcium,
g. fused silica, and
h. a composite of vermiculite, fumed silica, hardening agent, and drawn fiber.

16. A battery pack for selective attachment to a powered surgical instrument, the battery pack comprising a housing comprising two spaced walls forming a vacuum space therebetween, and at least one battery disposed in the housing, the vacuum space thermally insulating the battery.

17. The battery pack of claim 16 wherein the vacuum space is such that the life span of the battery is not significantly compromised when exposed to a temperature above its rated temperature.

18. The battery pack of claim 16 wherein the vacuum space is such that the life span of the battery is not significantly compromised when exposed to a temperature that is as much as 70 degrees C. above its rated temperature.

19. A battery pack for use with a medical instrument, the battery pack comprising a sealed enclosure placed under a vacuum, and at least one battery disposed in the housing, the vacuum thermally insulating the battery.

20. The battery pack of claim 19 wherein the vacuum is such that the life span of the battery is not significantly compromised when exposed toga temperature above its rated temperature.

21. The battery pack of claim 19 wherein the vacuum is such that the life span of the battery is not significantly compromised when exposed to a temperature that is as much as 70 degrees C. above its rated temperature.

22. A method of manufacturing a battery pack for use with a surgical instrument, the method comprising forming at least a portion of a housing of a thermal insulative material, and disposing at least one battery in the housing.

23. The method of claim 22 further comprising selecting the thermal insulative material so that the life span of the battery is not significantly compromised when exposed to a temperature above its rated temperature.

24. The method of claim 22 further comprising selecting the thermal insulative material so that the life span of the battery is not significantly compromised when exposed to a temperature that is as much as 70 degrees C. above its rated temperature.

25. The method of claim 22 further comprising selecting the thermal insulative material from the group consisting of:
a. a silica aerogel,
b. silicone chemical vapor deposition onto the surface of ceramic fabric,
c. fibers formed by a carbon, or silicon carbide, and oxide and impregnated with ceramic material,
d. a polymide foam,
e. a nanoporous silica coating on a polymer film,
f. a hydrous calcium,
g. fused silica, and
h. a composite of vermiculite, fumed silica, hardening agent, and drawn fiber.
